(19) United States
(12) Patent Application Publication
(10) Pub. No.: US 2009/0191171 A1
(43) Pub. Date: Jul. 30, 2009

(54) Reprogramming of Differentiated Progenitor or Somatic Cells Using Homologous Recombination

(76) Inventor: Yupo Ma, Las Vegas, NV (US)

Correspondence Address: DLA PIPER LLP (US), 4365 EXECUTIVE DRIVE, SUITE 1100, SAN DIEGO, CA, 92121-2133, US

(21) Appl. No.: 12/355,699

(22) Filed: Jan. 16, 2009

Related U.S. Application Data
(60) Provisional application No. 61/022,194, filed on Jan. 18, 2008.

(51) Int. Cl. A61K 35/12; C07H 21/04; C12N 15/63; C12N 15/87; C12Q 1/68; C12N 15/88; C12N 5/10

(52) U.S. Cl. 424/93.21; 536/24.1; 536/23.5; 435/320.1; 435/463; 435/6; 435/461; 435/458; 435/325

(57) ABSTRACT

The present invention provides methods and compositions for reprogramming somatic cells to a more primitive state, such as induced pluripotent stem cells, using homologous recombination. The induced pluripotent stem cells generated by the methods of the present invention are useful in a variety of therapeutic applications in the treatment and prevention of diseases and disorders.

### CROSS REFERENCE TO RELATED APPLICATION(S)

This application claims the benefit of priority under 35 U.S.C. § 119(e) of U.S. Ser. No. 61/022,194, filed Jan. 18, 2008, the entire content of which is incorporated herein by reference.

### BACKGROUND OF THE INVENTION

1. Field of the Invention

The invention relates generally to the genetic and epigenetic reprogramming of a differentiated cell using homologous recombination, and more specifically to reprogramming cells to confer a phenotype similar to progenitor cells of a given lineage or embryonic stem cells.

2. Background Information

Therapeutic uses of stem cells have been postulated since their isolation in 1998. However, several barriers exist before their potential can be utilized in human models. Among these barriers are both ethical issues and scientific issues. While ethical issues are complex and addressable only by political and religious consortia, scientific issues can be resolved with simple experiments. One major scientific obstacle that must be overcome prior to the use of stem cells therapeutically is the immune barrier. Previous attempts to avoid immune rejection have involved somatic cell nuclear transfer, a procedure that is technically challenging with extremely low efficiencies. In fact, the ethical implications far out weigh the therapeutic benefit for most people.

More recently, several published research accounts have reported the reprogramming of both mouse and human somatic (skin) cells to pluripotent cells, termed induced pluripotent stem (iPS) cells. These cells have great therapeutic potential because they can be tailored specifically to a patient or disease. In principle, an individual suffering from a genetic, degenerative, or malignant disorder could submit a skin biopsy for reprogramming to an iPS cell. Following reprogramming, a prescribed course of iPS cell differentiation to a specific tissue type could be initiated that would allow one to cure a given disorder. Proof of principle experiments have been done in mouse models. For example, mice displaying a phenotype similar to human sickle cell anemia were cured of the disease through somatic cell reprogramming and directed differentiation into blood cell progenitor populations. This is a clear demonstration of potential therapeutic uses for iPS cells.

While these experiments have been extremely promising, at least one major hurdle remains to be overcome, namely achieving the expression of certain genes required for reprogramming of somatic cells to iPS cells without incurring adverse consequences. Current studies have used retroviral delivery of the reprogramming genes into the genomic DNA, which may have deleterious effects because retroviral delivery causes random insertion of the reprogramming genes into the genome, raising the possibility that this delivery could insert into the coding sequence of a vital gene, blocking its expression. Not only this, but previously published reports have suggested that retroviral insertion occurs between 3-6 times for each gene. Depending on the number of genes introduced this could raise the number of insertions to 9 or more at random locations within the genome. While the probability of a deleterious retroviral insertion is quite low, this issue must be satisfactorily addressed before use in human subjects.

### SUMMARY OF THE INVENTION

The invention relates generally to the reprogramming of a differentiated or incompletely differentiated cell to a phenotype that is more primitive than that of the initial cell using homologous recombination. The invention contemplates a method for directing insertion of the gene or genes responsible for reprogramming the somatic cell by homologous recombination such that the site of insertion within the genome is a pre-determined insertion site and such that the insertion event does not have an adverse effect upon the recipient cell.

Accordingly, the invention provides a nucleic acid construct for targeted delivery of genes capable of inducing pluripotency in a somatic cell through homologous recombination with the genome of the somatic cell, such that the nucleic acid is directed to a pre-determined insertion site in the genome that will not result in adverse effects upon the recipient somatic cell. The nucleic acid construct includes, in 5′ to 3′ orientation, a first polynucleotide sequence capable of homologous recombination with a first region of a target polynucleotide sequence, a second polynucleotide sequence encoding an expression cassette including at least one gene that induces pluripotency, and a third polynucleotide sequence capable of homologous recombination with a second region of the target polynucleotide sequence. The expression cassette further includes in operable linkage to the gene that induces pluripotency a promoter and a translation initiation site. In various aspects, the expression cassette further includes a selectable marker, such as a lethal gene. In a related aspect, the gene or genes capable of inducing pluripotency may be one or more of a SOX family gene, a KLF family gene, a MYC family gene, SALL4, OCT4, NANOG, LIN28, NOBOX, STELLA, Esrrb or a STAT family gene. STAT family members may include, for example STAT1, STAT2, STAT3, STAT4, STAT5 (STAT5A and STAT5B), and STAT6. In an exemplary aspect, the cassette includes four genes capable of inducing pluripotency, such as OCT4, SOX2, KLF4 and C-MYC, wherein a translation initiation site is spaced between each of the genes.

In another embodiment, the invention provides a vector including a nucleic acid construct for targeted delivery of genes capable of inducing pluripotency in a somatic cell through homologous recombination with the genome of the somatic cell. The nucleic acid construct includes, in 5′ to 3′ orientation, a first polynucleotide sequence capable of homologous recombination with a first region of a target polynucleotide sequence, a second polynucleotide sequence encoding an expression cassette including at least one gene that induces pluripotency, and a third polynucleotide sequence capable of homologous recombination with a second region of the target polynucleotide sequence. The expression cassette further includes in operable linkage a promoter and a translation initiation site. In various aspects, the expression cassette further includes a selectable marker, such as a lethal gene.

In another embodiment, the invention provides a method of generating an induced pluripotent stem (iPS) cell. The method includes introducing a nucleic acid construct of the present invention into a somatic cell. Introduction of the construct into the somatic cell allows integration of the construct into the somatic cell genome through homologous recombination and expression of at least one gene that induces pluripotency, thereby reprogramming the somatic cell and generating an induced pluripotent stem (iPS) cell. In one aspect of the invention, the introduction and integration of the nucleic acid construct into the somatic cell is performed using a non-viral based transfection technique. In an exemplary aspect, integration of the construct results from targeted homologous recombination with introduction of the construct into the genome of the host cell using a non-viral-mediated transfer technique, such as, electroporation, calcium phosphate mediated transfer, nucleofection, sonoporation, heat shock, magnetofection, liposome mediated transfer, microinjection, microprojectile mediated transfer (nanoparticles), cationic polymer mediated transfer, or cell fusion.

In another embodiment, the present invention provides an induced pluripotent stem (iPS) cell produced using the methods described herein.

In another embodiment, the present invention provides a population of induced pluripotent stem (iPS) cells produced using the methods described herein.

In another embodiment, the present invention provides a method of treating a subject with induced pluripotent stem (iPS) cells. The method includes obtaining a somatic cell from a subject, reprogramming the somatic cell into an induced pluripotent stem (iPS) cell using the methods described herein, culturing the iPS cell under conditions that allow the iPS cell to differentiate into a desired cell type suitable for treating a condition, and introducing into the subject the differentiated cell, thereby treating the condition.

### BRIEF DESCRIPTION OF THE DRAWINGS

FIG. 1 is an illustrative drawing of a nucleic acid construct including a single gene of interest that may be introduced via targeted homologous recombination into the genome of a somatic cell. The construct includes an expression cassette including a promoter, the gene of interest, and a drug resistance gene.

FIG. 2 is an illustrative drawing of a homologous reprogramming cassette for somatic cell reprogramming. The cassette is configured for targeted integration into genomic DNA (gDNA) at the SALL4 locus by incorporation of flanking gDNA sequences capable of homologous recombination and integration at the SALL4 locus target. The construct includes a cytolomegalovirus (CMV) promoter which is a constitutively active promoter in most cell types and used to regulate transcription of SALL4. The construct further includes a translation initiation site (an internal ribosome entry site or IRES) which is used to regulate expression of the drug resistance gene (Neomycin) from the CMV promoter.

FIG. 3 is an illustrative drawing of a nucleic acid construct including multiple genes of interest that may be introduced via targeted homologous recombination into the genome of a somatic cell. The construct includes an expression cassette including multiple genes of interest under the control of a promoter.

FIG. 4 is an illustrative drawing of the cloning strategy used for construction of a targeting vector using the Gateways Cloning System. The system allows three different vectors to be used in a recombination reaction that correctly and specifically orients each arm in the final targeting vector.

FIG. 5 is an illustrative drawing of a nucleic acid construct generated to reprogram somatic cells using a homologous recombination approach for targeted integration at the OCT4 loci of the host cell genome.

### DETAILED DESCRIPTION OF THE INVENTION

The present invention is based on innovative nucleic acid constructs and an approach involving homologous recombination to reprogram differentiated or semi-differentiated cells to a phenotype that is more primitive than that of the initial cell.

Before the present composition, methods, and treatment methodology are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, an and “the” include plural references unless the context clearly dictates otherwise. Thus, for example, references to “the method” includes one or more methods, and/or steps of the type described herein which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described.

The present invention provides an approach involving homologous recombination to reprogram differentiated or incompletely differentiated cells to a phenotype that is more primitive than that of the initial cell without requiring retroviral delivery. This may include, but is not limited to, the introduction of promoter regions (be they activating, inducible, or inhibiting) upstream of endogenous genes, the introduction of drug selection cassettes, or the introduction of entire expression cassettes that include not only promoter regions but also the coding sequences for one or more genes using homologous recombination for the purpose of reprogramming cells to confer a phenotype similar to progenitor cells of a given lineage (as a non-limiting example, hematopoietic stem cells) or of embryonic stem cells.

Accordingly, the present invention is based on the innovative concept of reprogramming somatic or progenitor cells into iPS cells using homologous recombination. Through recombination, it is possible to introduce reprogramming genes into defined regions on the chromosomes, avoiding random insertions. The recombination sites can also be sequenced to validate their exact genomic location and thus provide a much safer avenue for in vivo use. Following reprogramming, differentiation into specific tissues is then possible for a variety of therapeutic purposes.

As used herein, pluripotent cells include cells that have the potential to divide in vitro for an extended period of time (greater than one year) and have the unique ability to differentiate into cells derived from all three embryonic germ layers, namely endoderm, mesoderm and ectoderm.

Somatic cells for use with the present invention may be primary cells or immortalized cells. Such cells may be primary cells (non-immortalized cells), such as those freshly isolated from an animal, or may be derived from a cell line (immortalized cells). In an exemplary aspect, the somatic cells are mammalian cells, such as, for example, human cells or mouse cells. They may be obtained by well-known methods, from different organs, such as, but not limited to skin, lung, pancreas, liver, stomach, intestine, heart, reproductive organs, bladder, kidney, urethra and other urinary organs, or generally from any organ or tissue containing living somatic cells, or from blood cells. Mammalian somatic cells useful in the present invention include, by way of example, adult stem cells, sertoli cells, endothelial cells, granulosa epithelial cells, neurons, pancreatic islet cells, epidermal cells, epithelial cells, hepatocytes, hair follicle cells, keratinocytes, hematopoietic cells, melanocytes, chondrocytes, lymphocytes (B and T lymphocytes), erythrocytes, macrophages, monocytes, mononuclear cells, fibroblasts, cardiac muscle cells, other known muscle cells, and generally any live somatic cells. In particular embodiments, fibroblasts are used. The term somatic cell, as used herein, is also intended to include adult stem cells. An adult stem cell is a cell that is capable of giving rise to all cell types of a particular tissue. Exemplary adult stem cells include hematopoietic stem cells, neural stem cells, and mesenchymal stem cells.

Homologous recombination itself is a rather common occurrence during the process of meiosis in eukaryotic systems. The process involves the alignment of highly similar DNA sequences in chromosomes, and the exchange of DNA sequences between the DNA in each of the sister chromosomes. The complex series of molecular interactions is simply defined as “cross-over”. When these sequences are aligned, breaks in the double strand of DNA can facilitate the swapping of genetic material. Designed correctly, it is possible to use two homologous sequences flanking a non-homologous sequence to introduce a foreign DNA fragment to the genomic DNA. This strategy has been used extensively for gene knock-in or knock-out in mice. While there are several potential advantages of this system, a key advantage is the elimination of the need for retroviral delivery of genes necessary for reprogramming somatic cells. Further, because homologous recombination requires highly similar stretches of DNA sequence, relative certainty is afforded of the location on the delivered insert and of the copy number (one or two as compared with 3-6 for retroviral delivery).

There are several possible avenues to achieve successful generation of induced pluripotent stem (iPS) cells from somatic cells. First, it is possible to introduce a foreign promoter that is continually active, inducible, or inhibitory. This allows for expression or inhibition of genes that are necessary to reprogram the somatic cell to an iPS cell. However, this does not allow for selection of cells that homologously recombined due to lack of a selectable marker, such as a drug resistance marker.

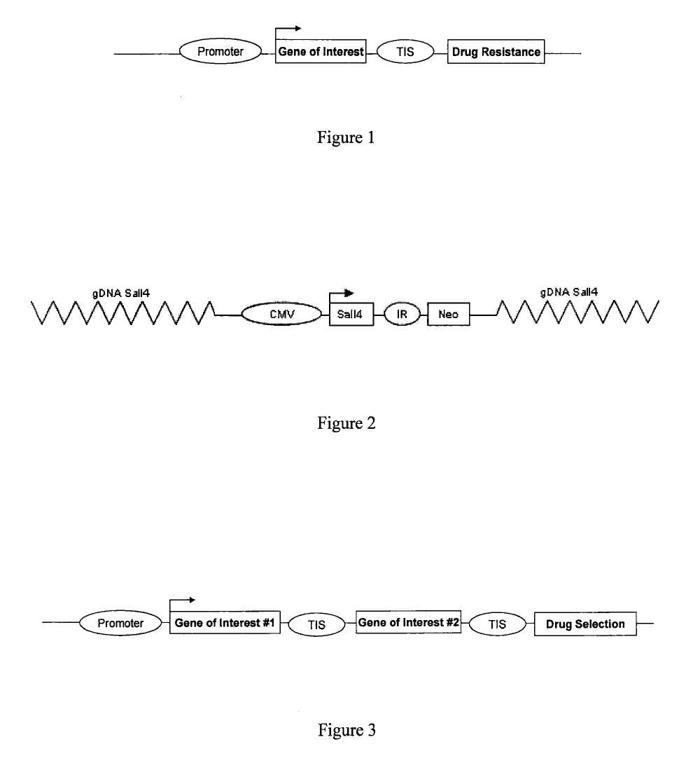

Alternatively, successful generation of iPS cells is possible through introduction of an expression cassette consisting of a promoter, a gene or genes that induce(s) pluripotency, and a selectable marker, such as a drug resistance gene. The gene of interest and drug resistance gene are preferably separated by a translation initiation site (TIS), such as for example, an internal ribosome entry site (IRES), to allow for expression of both genes to be preferably controlled by the same promoter (FIG. 1). By expressing the gene of interest from the cassette it is possible to reprogram the somatic cell into an iPS cell. For example, using homologous recombination it is possible to introduce into the SALL4 loci (FIG. 2) of a somatic cell genome an expression cassette consisting of a CMV promoter, the coding sequence for the gene SALL4 (a gene associated with pluripotency and somatic cell reprogramming), and a gene encoding resistance to the drug neomycin. It should be understood, that various isoforms of SALL4 are included in the invention. These include but are not limited to SALL1, SALL2, SALL3, and SALL4 as well as SALL4 mRNA spliced forms, SALL4A and SALL4B.

Another alternative would be to construct a single insertion cassette of multiple genes and selection markers for homologous recombination. By combining the coding sequences of many genes end to end, one could ideally reprogram a cell to an iPS cell with one insertion. In this system, a promoter would drive expression of the string of genes of interest separated by translation initiation sites as shown in the construct of FIG. 3. The advantages of this system are that it allows the construction of a cassette containing several coding sequences that can be inserted into the genome in a correctly oriented and specific site, and that requires only one homologous recombination event and therefore only one drug selection.

Accordingly, in one aspect, the invention provides a nucleic acid construct for targeted delivery of genes capable of inducing pluripotency in a somatic cell through homologous recombination with the genome of the somatic cell. The nucleic acid construct includes in 5′ to 3′ orientation, a first polynucleotide sequence capable of homologous recombination with a first region of a target polynucleotide sequence, a second polynucleotide sequence encoding an expression cassette including at least one gene that induces pluripotency, and a third polynucleotide sequence capable of homologous recombination with a second region of the target polynucleotide sequence. The expression cassette further includes in operable linkage a promoter, at least one gene that induces pluripotency, and a translation initiation site.

As used herein, the term “operatively linked” means that two or more molecules are positioned with respect to each other such that they act as a single unit and effect a function attributable to one or both molecules or a combination thereof. For example, a polynucleotide encoding a gene can be operatively linked to a transcriptional or translational regulatory element, in which case the element confers its regulatory effect on the polynucleotide similar to the way in which the regulatory element would effect a polynucleotide sequence with which it normally is associated with in a cell.

The term “polynucleotide” or “nucleotide sequence” or “nucleic acid molecule” is used broadly herein to mean a sequence of two or more deoxyribonucleotides or ribonucleotides that are linked together by a phosphodiester bond. As such, the terms include RNA and DNA, which can be a gene or a portion thereof, a cDNA, a synthetic polydeoxyribonucleic acid sequence, or the like, and can be single stranded or double stranded, as well as a DNA/RNA hybrid. Furthermore, the terms as used herein include naturally occurring nucleic acid molecules, which can be isolated from a cell, as well as synthetic polynucleotides, which can be prepared, for example, by methods of chemical synthesis or by enzymatic methods such as by the polymerase chain reaction (PCR). It should be recognized that the different terms are used only for convenience of discussion so as to distinguish, for example, different components of a composition.

In general, the nucleotides comprising a polynucleotide are naturally occurring deoxyribonucleotides, such as adenine, cytosine, guanine or thymine linked to 2′-deoxyribose, or ribonucleotides such as adenine, cytosine, guanine or uracil linked to ribose. Depending on the use, however, a polynucleotide also can contain nucleotide analogs, including non-naturally occurring synthetic nucleotides or modified naturally occurring nucleotides. Nucleotide analogs are well known in the art and commercially available, as are polynucleotides containing such nucleotide analogs. The covalent bond linking the nucleotides of a polynucleotide generally is a phosphodiester bond. However, depending on the purpose for which the polynucleotide is to be used, the covalent bond also can be any of numerous other bonds, including a thiodiester bond, a phosphorothioate bond, a peptide-like bond or any other bond known to those in the art as useful for linking nucleotides to produce synthetic polynucleotides.

A polynucleotide comprising naturally occurring nucleotides and phosphodiester bonds can be chemically synthesized or can be produced using recombinant DNA methods, using an appropriate polynucleotide as a template. In comparison, a polynucleotide comprising nucleotide analogs or covalent bonds other than phosphodiester bonds generally will be chemically synthesized, although an enzyme such as T7 polymerase can incorporate certain types of nucleotide analogs into a polynucleotide and, therefore, can be used to produce such a polynucleotide recombinantly from an appropriate template.

In various aspects of the present invention, genes that induce pluripotency are utilized to reprogram differentiated or incompletely differentiated cells to a phenotype that is more primitive than that of the initial cell, such as the phenotype of an iPS cell. Such genes are capable of generating an iPS cell from a somatic cell upon expression of one or more such genes having been integrated into the genome of the somatic cell. As used herein, a gene that induces pluripotency is intended to refer to a gene that is associated with pluripotency and capable of generating a less differentiated cell, such as an iPS cell from a somatic cell upon integration and expression of the gene. The expression of a pluripotency gene is typically restricted to pluripotent stem cells, and is crucial for the functional identity of pluripotent stem cells.

Several genes have been found to be associated with pluripotency and suitable for use with the present invention. Such genes are known in the art and include, by way of example, SOX family genes (SOX1, SOX2, SOX3, SOX15, SOX18), KLF family genes (KLF1, KLF2, KLF4, KLF5), MYC family genes (C-MYC, L-MYC, N-MYC), SALL4, OCT4, NANOG, LIN28, STELLA, NOBOX Esrrb or a STAT family gene. STAT family members may include, for example STAT1, STAT2, STAT3, STAT4, STAT5 (STAT5A and STAT5B), and STAT6, FoxD3, UTF1, Rex1, ZNF206, Myb12, DPPA2, ESG1, Otx2 and combinations thereof. While in some instances, use of only one gene to induce pluripotency may be possible, in general, expression of more than one gene is required to induce pluripotency. For example, two, three, four or more genes may be simultaneously integrated into the somatic cell genome as a polycistronic construct to allow simultaneous expression of such genes. In an illustrative aspect, four genes are utilized to induce pluripotency including OCT4, SOX2, KLF4 and C-MYC. It has been shown previously that as few as two factors may be sufficient to reprogram somatic cells, e.g., using OCT4 and SOX2, however, as few as one factor may be sufficient to reprogram the cells. Preferably, the potency-determining factor may be a transcription factor and may include other factors known in the art.

The term “nucleic acid construct” or “recombinant nucleic acid molecule” is used herein to refer to a polynucleotide that is manipulated by human intervention. A recombinant nucleic acid molecule can contain two or more nucleotide sequences that are linked in a manner such that the product is not found in a cell in nature. In particular, the two or more nucleotide sequences can be operatively linked and, for example, can encode multiple genes, such as genes that induce pluripotency, along with regulatory elements for controlling expression of such genes.

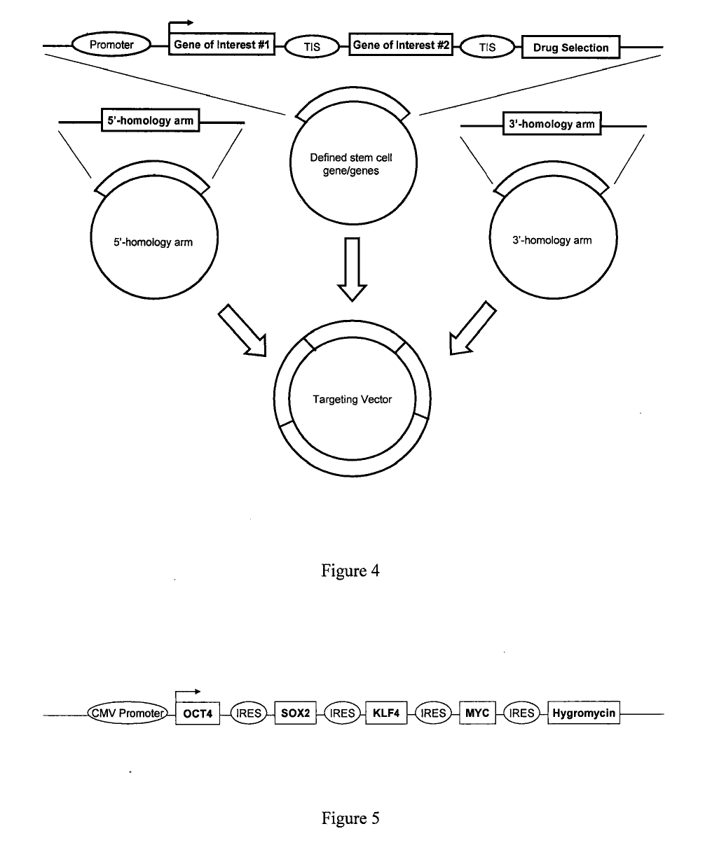

A discussed herein, one advantage of utilizing homologous recombination for integration of the engineered nucleic acid construct of the present invention is that homologous recombination allows for targeted integration of the construct. Successful targeting of the insertion site can facilitate expression of the inserted genes under appropriate circumstances and/or avoid inactivation of a vital gene as a result of a random insertion event. For homologous recombination to occur, the nucleic acid construct includes polynucleotides homologous to the targeted region of the genome of the host cell to allow a “crossover” event to occur. Accordingly, the nucleic acid construct of the present invention includes polynucleotide sequences flanking (i.e., upstream and downstream) the expression cassette including the genes that induce pluripotency, that allow for homologous recombination to occur. As shown in FIG. 4, the construct includes a first polynucleotide sequence (e.g. the 5′ homology arm) capable of homologous recombination with a first region of a target polynucleotide sequence and a third polynucleotide sequence (e.g., the 3′ homology arm) capable of homologous recombination with a second region of the target polynucleotide sequence. The first and third polynucleotide sequences are homologous to a first and second region of the target polynucleotide sequence, such as a region in a somatic cell genome. Accordingly, the sequences can include a nucleotide sequence of somatic cell genomic DNA (gDNA) that is sufficient to undergo homologous recombination with somatic cell genomic DNA, for example, a nucleotide sequence comprising about 400 to 5000 or more substantially contiguous nucleotides of somatic cell genomic DNA. In various embodiments, the nucleic acid construct may be configured for homologous recombination with any locus or loci within a somatic cell genome, such as the OCT4 and/or SALL4 locus or locis.

In various aspects, the second polynucleotide encoding the expression cassette of the nucleic acid construct of the present invention further includes a selectable marker, such as, a lethal gene. For example, the expression cassette includes one or more genes that induce pluripotency in operable linkage with a selectable marker. Accordingly, in various embodiments, the one or more genes that induce pluripotency may be co-expressed with the selectable marker. As such, cells that are reprogrammed as a result of expression of the genes that induce pluripotency will also express a selectable phenotype determined by the selectable marker employed. For example, in various embodiments, the selectable marker may be a gene that confers drug resistance. Thus, reprogrammed cells may be easily identified by their selectable marker and may be selectively grown and proliferated while non-reprogrammed cells will be eliminated.

Thus, a selectable marker, as used herein, is a marker that, when expressed, confers upon a cell a selectable phenotype, such as, but not limited to, antibiotic resistance, resistance to a cytotoxic agent, nutritional prototrophy or expression of a surface protein. Co-expression of the selectable marker and one or more genes that induce pluripotency make it possible to identify and select reprogrammed cells in which the integrated pluripotency genes are expressed. A variety of selectable marker genes are suitable for use with the present invention including, but not limited to, the neomycin resistance gene, puromycin resistance gene, guanine phosphoribosyl transferase, dihydrofolate reductase, adenosine deaminase, puromycin-N-acetyltransferase, hygromycin resistance gene, multi-drug resistance gene, and hisD gene.

In various aspects, the second polynucleotide encoding the expression cassette of the nucleic acid construct of the present invention further includes one or more promoters. As used herein, a promoter is intended mean a polynucleotide sequence capable of facilitating transcription of genes in operable linkage with the promoter. While several types of promoters are well known in the art and suitable for use with the present invention, in an exemplary aspect the promoter is a constitutive promoter that allows for unregulated expression in mammalian cells, such as the cytomegalovirus (CMV) promoter.

Alternatively, the exogenously introduced genes that induce pluripotency may be expressed from one or more inducible promoters. An inducible promoter is a promoter that, in the absence of an inducer (such as a chemical and/or biological agent), does not direct expression, or directs low levels of expression of an operably linked gene (including cDNA), and, in response to an inducer, its ability to direct expression is enhanced. Exemplary inducible promoters include, for example, promoters that respond to heavy metals, to thermal shocks, to hormones, and those that respond to chemical agents, such as glucose, lactose, galactose or antibiotic.

In various related aspects, the second polynucleotide encoding the expression cassette of the nucleic acid construct of the present invention further includes one or more TIS (e.g., IRES). Where multiple genes are included in the expression cassette, a TIS is ideally positioned between each gene to allow each gene to be driven off of a single upstream promoter.

A nucleic acid construct useful in a method of the invention can be contained in a vector. One potential drawback of generating the nucleic acid constructs of the present invention is the construction of the targeting vector. The homologous recombination step requires flanking DNA that is identical in sequence to the targeted locus, and a positive selection marker (e.g., antibiotic resistance). Accordingly, the vector can be any vector useful for introducing a nucleic acid construct of the present invention into a somatic cell.

Because of advances in cloning technology, individuals familiar with the art can with relative ease construct the cloning vectors containing the reprogramming genes and other sequences to be inserted. For example, Gateway® cloning technology, developed by Invitrogen Inc., enables the orienting and insertion of multiple polynucleotide fragments into a target vector in one step which is suitable for homologous recombination (FIG. 4).

The present invention further provides a method of generating an iPS cell. Generally, the method includes introducing a nucleic acid construct of the present invention into a somatic cell to allow for integration and expression of genes that induce pluripotency to reprogram the somatic cell to an undifferentiated or less differentiated state. Introduction of the construct into the somatic cell allows integration of the construct into the somatic cell genome through homologous recombination and expression of the at least one gene that induces pluripotency, thereby reprogramming the somatic cell and generating an iPS cell.

Traditionally, viral-mediated techniques that do not utilize targeted homologous recombination have been used to introduce and integrate genes involved with pluripotency into a somatic cell genome. However, use of viral-mediated techniques have several disadvantages including non-targeted integration into the host genome. Accordingly, in the present method, the introduction and integration of the nucleic acid construct into the somatic cell is performed using a non-viral based technique. In an exemplary aspect, the method incorporates targeted integration of the nucleic acid construct of the present invention via homologous recombination with the host genome.

The nucleic acid construct of the present invention may be introduced into a cell using a variety of well known techniques, such as non-viral based transfection of the cell. In an exemplary aspect the construct is incorporated into a vector and introduced into the cell to allow homologous recombination. Introduction into the cell may be performed by any non-viral based transfection known in the art, such as, but not limited to, electroporation, calcium phosphate mediated transfer, nucleofection, sonoporation, heat shock, magnetofection, liposome mediated transfer, microinjection, microprojectile mediated transfer (nanoparticles), cationic polymer mediated transfer (DEAE-dextran, polyethylenimine, polyethylene glycol (PEG) and the like) or cell fusion. Other methods of transfection include proprietary transfection reagents such as Lipofectamine™, Dojindo Hilymax™, Fugene™, jetPEI™, Effectene™ and DreamFect™.

As used herein, reprogramming, is intended to refer to a process that alters or reverses the differentiation status of a somatic cell that is either partially or terminally differentiated. Reprogramming of a somatic cell may be a partial or complete reversion of the differentiation status of the somatic cell. In an exemplary aspect, reprogramming is complete wherein a somatic cell is reprogrammed into an induced pluripotent stem cell. However, reprogramming may be partial, such as reversion into any less differentiated state. For example, reverting a terminally differentiated cell into a cell of a less differentiated state, such as a multipotent cell.

As discussed herein, expression of the exogenously introduced genes that induce pluripotency simultaneously with a selectable marker allows for rapid identification of reprogrammed cells. Accordingly, the methods of the present invention further include detecting the selectable marker. Reprogrammed cells may be easily identified by their selectable marker and may be selectively grown and proliferated while non-reprogrammed cells will perish.

Further analysis may be performed to assess the pluripotent characteristics of a reprogrammed cell. The cells may be analyzed for different growth characteristics and embryonic stem cell like morphology. For example, cells may be differentiated in vitro by adding certain growth factors known to drive differentiation into specific cell types. Reprogrammed cells capable of forming only a few cell types of the body are multipotent, while reprogrammed cells capable of forming any cell type of the body are pluripotent. Expression profiling of reprogrammed somatic cells to assess their pluripotency characteristics may also be conducted. Expression of individual genes associated with pluripotency may also be examined. Additionally, expression of embryonic stem cell surface markers may be analyzed. Detection and analysis of a variety of genes known in the art to be associated with pluripotent stem cells may include analysis of genes such as, but not limited to OCT4, NANOG, SALL4, SSEA-1, SSEA-3, SSEA-4, TRA-1-60, TRA-1-81, or a combination thereof.

The invention further provides iPS cells produced using the methods described herein, as well as populations of such cells. The reprogrammed cells of the present invention, capable of differentiation into a variety of cell types, have a variety of applications and therapeutic uses. The basic properties of stem cells, the capability to infinitely self-renew and the ability to differentiate into every cell type in the body make them ideal for therapeutic uses.

Accordingly, in one aspect the present invention further provides a method of treatment or prevention of a disorder and/or condition in a subject using iPS cells generated using the methods described herein. The method includes obtaining a somatic cell from a subject and reprogramming the somatic cell into an iPS cell using the methods described herein. The cell is then cultured under suitable conditions to differentiate the cell into a desired cell type suitable for treating the condition. The differentiated cell may then be introduced into the subject to treat or prevent the condition.

One advantage of the present invention is that it provides an essentially limitless supply of isogenic or syngenic human cells suitable for transplantation. The iPS cells are tailored specifically to the patient, avoiding immune rejection. Therefore, it will obviate the significant problem associated with current transplantation methods, such as, rejection of the transplanted tissue which may occur because of host versus graft or graft versus host rejection. For example, use of iPS cells of the present invention in bone marrow transplants, will circumvent the requirement of providing heavy immune suppression with drugs that have potentially adverse side effects to avoid rejection.

The iPS cells of the present invention may be differentiated into a number of different cell types to treat a variety of disorders by methods known in the art. For example, iPS cells may be induced to differentiate into hematopoetic stem cells, muscle cells, cardiac muscle cells, liver cells, cartilage cells, epithelial cells, urinary tract cells, neuronal cells, and the like. The differentiated cells may then be transplanted back into the patient's body to prevent or treat a condition.

The methods of the present invention can also be used in the treatment or prevention of neurological diseases. Such diseases include, for example, Alzheimer's disease, Parkinson's disease, Huntington's disease, amyotrophic lateral sclerosis (ALS), lysosomal storage diseases, multiple sclerosis, spinal cord injuries and the like.

The methods of the present invention can also be used to correct mutations of single genes. These mutations account for diseases such as cystic fibrosis, hemophilia, and various cancers such as those associated with the BRCA1 and BRCA2 mutations with high risk of development of breast and ovarian cancers.

The cells produced in the methods of the invention can be utilized for repairing or regenerating a tissue or differentiated cell lineage in a subject. The method includes obtaining the reprogrammed cell as described herein and administering the cell to a subject (e.g., a subject having a myocardial infarction, congestive heart failure, stroke, ischemia, peripheral vascular disease, alcoholic liver disease, cirrhosis, Parkinson's disease, Alzheimer's disease, diabetes, cancer, arthritis, wound healing, immunodeficiency, aplastic anemia, anemia, and genetic disorders) and similar diseases, where an increase or replacement of a particular cell type/tissue or cellular de-differentiation is desirable. In one embodiment, the subject has damage to the tissue or organ, and the administering provides a dose of cells sufficient to increase a biological function of the tissue or organ or to increase the number of cell present in the tissue or organ. In another embodiment, the subject has a disease, disorder, or condition, and wherein the administering provides a dose of cells sufficient to ameliorate or stabilize the disease, disorder, or condition. In yet another embodiment, the subject has a deficiency of a particular cell type, such as a circulating blood cell type and wherein the administering restores such circulating blood cells.

In one aspect of this invention, a single gene is used to effect cell reprogramming to ease the clinical transition of iPS cells. In a non-limiting example described herein, the single gene is SALL4. The genetic integration of a single gene into the host genome significantly reduces the complications associated with genetic reactivation and/or insertional mutagenesis currently encountered in the field.

The following examples are provided to further illustrate the advantages and features of the present invention, but are not intended to limit the scope of the invention. While they are typical of those that might be used, other procedures, methodologies, or techniques known to those skilled in the art may alternatively be used.

### Example 1

### Generation of a Polycistronic Vector Construct Suitable for Homologous Recombination

This example illustrates the generation of a polycistronic vector construct including four genes that induce pluripotency suitable for targeted integration into a somatic cell genome via homologous recombination.

The present example illustrates the design and execution of homologous recombination based cellular retrodifferentation for therapeutic purposes. Recent research has suggested that the genes OCT4, SOX2, KLF4, and c-MYC are able to reprogram fetal fibroblast cells to confer a stem cell-like phenotype. However, as discussed herein, other genes may also be utilized to reprogram somatic and progenitor cells using a similar vector design. Classical cloning techniques were used to design and create a fragment of these four genes driven by the cytomegalovirus (CMV) promoter and separated by an internal ribosomal entry site (IRES). The partial expression cassette is shown in FIG. 4.

The CMV promoter drives expression of nearly any gene of interest in eukaryotic systems while IRES allows for translation of multiple proteins driven from one promoter by serving as a type of translation initiation site. This method was utilized primarily because it only requires the insertion of exogenous sequence into one loci, and therefore, requires only one drug selection (in this case hygromycin). Selecting the endogenous loci just downstream of the transcription start site of OCT4, present in the genomic DNA, for our targeted insertion point, both the 5′-homology arm, at a length of 3.5 kb, and the 3′-homology arm, at 2.6 kb, were successfully cloned. The final target vector construct was made using the 4-way recombination mediated by the Gateway® Cloning System as shown in FIG. 4.

### Example 2

### Reprogramming of Somatic Cells Using a SALL4 Expression Construct Integrated Via Homologous Recombination

The following This example illustrates the reprogramming of somatic cells by integration of a construct expressing endogenous SALL4 via homologous recombination.

Focusing intensely on the role of SALL4 in embryonic stem cells the targeting construct shown in FIG. 2 was generated. It has been previously shown that SALL4 regulates the expression of vital reprogramming factors in embryonic stem cells and thus, implicated in somatic cell reprogramming.

Following generation of the CMV-SALL4-neo targeting construct the plasmid was electroporated into mouse tail tip fibroblasts expressing SALL4-GFP promoter-reporter construct. A SALL4 expression cassette was integrated into the SALL4 locus of the genomic DNA using homologous recombination because heterozygous SALL4 mice have no obvious phenotype. After 17 days post transfection (10 days in ES media), ES-like clones expressed very low level of green fluorescent protein (GFP) indicating incomplete reprogramming at this stage. Surrounding fibroblasts did not express GFP serving as the negative control. The phase contrast images showed fibroblast cells and a potential iPS cell colony. The expression of SALL4 within the potential iPS cell colony is suggestive of pluripotency. After 22 days post transfection (15 days in ES media), ES-like clones highly expressed GFP, indicating complete reprogramming at this stage. The phase contrast images allowed identification of fibroblast cells under different magnifications and a potential iPS cell colony expressing GFP under different magnifications. Surrounding fibroblasts did not express GFP serving as the negative control. The results indicate that SALL4 alone may be capable of reprogramming mouse fibroblast cells to pluripotency via introduction by homologous recombination.

Expression was examined after 10 days culture in mES media of SALL4 (promoter)-GFP (reporter) constructs in tail-tip fibroblasts (TTFs) including CMV-SALL4-neo expression cassettes integrated via homologous recombination at the SALL4 loci. Phase contrast images showed fibroblast cells and a induced pluripotent stem (iPS) cell colony. Green fluorescent protein (GFP) production was used to indicate SALL4 expression. The level of SALL4 expression within the iPS cell colony is suggestive of pluripotency.

Studies were done to show somatic cell reprogramming using overexpression of a single transcription factor, SALL4, by homologous recombination. Expression was shown after 15 days culture in mES media of SALL4 (promoter)-GFP (reporter) constructs in tail-tip fibroblasts (TTFs) including CMV-SALL4-neo expression cassettes integrated via homologous recombination at the SALL4 loci. Phase contrast images showed fibroblast cells and a potential induced pluripotent stem (iPS) cell colony under different magnifications. GFP expression was also examined under different magnifications. Surrounding fibroblasts did not express GFP serving as the negative control. ES-like clones highly expressed GFP indicating reprogramming.

Although the invention has been described with reference to the above example, it will be understood that modifications and variations are encompassed within the spirit and scope of the invention. Accordingly, the invention is limited only by the following claims.

What is claimed is:

1. A nucleic acid construct comprising in 5′ to 3′ orientation:
a) a first polynucleotide sequence capable of homologous recombination with a first region of a target polynucleotide sequence;
b) a second polynucleotide sequence encoding an expression cassette in operable linkage comprising in 5′ to 3′ orientation:
i) a promoter;
ii) at least one gene that induces pluripotency; and
iii) a translation initiation site; and
c) a third polynucleotide sequence capable of homologous recombination with a second region of the target polynucleotide sequence.

2. The nucleic acid construct of claim 1, wherein the expression cassette comprises two or more genes that induce pluripotency.

3. The nucleic acid construct of claim 2, wherein a translation initiation site is spaced between each gene that induces pluripotency.

4. The nucleic acid construct of claim 1, wherein the at least one gene is a SOX family gene, a KLF family gene, a MYC family gene, SALL4, OCT4, NANOG, or LIN28.

5. The nucleic acid construct of claim 4, wherein the at least one gene is selected from the group consisting of: SOX1, SOX2, SOX3, SOX15, SOX18, KLF1, KLF2, KLF4, KLF5, C-MYC, L-MYC, N-MYC, SALL4, OCT4, NANOG, STELLA, Esrrb, NOBOX STAT family members FoxD3, UTF1, Rex1, ZNF206, Myb12, DPPA2, ESG1, Otx2 and LIN28, and any combination thereof.

6. The nucleic acid construct of claim 2, wherein the expression cassette comprises four genes that induce pluripotency.

7. The nucleic acid construct of claim 6, wherein the genes are OCT4, SOX2, KLF4 and C-MYC.

8. The nucleic acid construct of claim 1, wherein the expression cassette further comprises a selectable marker.

9. The nucleic acid construct of claim 1, wherein the selectable marker is a gene selected from the group consisting of: neomycin resistance gene, puromycin resistance gene, guanine phosphoribosyl transferase, dihydrofolate reductase, adenosine deaminase, puromycin-N-acetyltransferase, hygromycin resistance gene, multidrug resistance gene, or hisD gene.

10. The nucleic acid construct of claim 9, wherein the selectable marker is the hygromycin resistance gene.

11. The nucleic acid construct of claim 1, wherein the first and third polynucleotide sequences have a length of between about 0.5 kb and 5 kb.

12. The nucleic acid construct of claim 11, wherein the first polynucleotide sequence has a length of about 3.5 kb.

13. The nucleic acid construct of claim 11, wherein the third polynucleotide sequence has a length of about 2.6 kb.

14. The nucleic acid construct of claim 1, wherein the promoter is a cytomegalovirus (CMV) promoter.

15. The nucleic acid construct of claim 1, wherein the translation initiation site is an internal ribosome entry site (IRES).

16. A vector comprising the construct of claim 1.

17. A method of generating an induced pluripotent stem (iPS) cell comprising:
a) introducing a nucleic acid construct into a somatic cell, wherein the construct comprises in 5′ to 3′ orientation:
i) a first polynucleotide sequence capable of homologous recombination with a first region of a target polynucleotide sequence of the somatic cell genome;
ii) a second polynucleotide sequence encoding an expression cassette in operable linkage comprising in 5′ to 3′ orientation, a promoter, at least one gene that induces pluripotency, and a translation initiation site; and
iii) a third polynucleotide sequence capable of homologous recombination with a second region of the target polynucleotide sequence of the somatic cell genome; wherein introduction of the construct into the somatic cell allows integration of the construct into the somatic cell genome through homologous recombination and expression of the at least one gene, thereby reprogramming the somatic cell and generating an induced pluripotent stem (iPS) cell.

18. The method of claim 17, wherein the expression cassette further comprises a selectable marker.

19. The method of claim 18, further comprising detecting the selectable marker.

20. The method of claim 17, further comprising detecting a pluripotent stem cell marker after expression of the at least one gene.

21. The method of claim 20, wherein the pluripotent stem cell marker is selected from OCT4, NANOG, SALL4, SSEA-1, SSEA-3, SSEA-4, TRA-1-60, TRA-1-81, or a combination thereof.

22. The method of claim 17, wherein the expression cassette comprises two or more genes that induce pluripotency.

23. The method of claim 22, wherein a translation initiation site is spaced in operable linkage between each gene that induces pluripotency.

24. The method of claim 17, wherein the at least one gene is a SOX family gene, a KLF family gene, a MYC family gene, SALL4, OCT4, NANOG, or LIN28.

25. The method of claim 24, wherein the gene is selected from the group consisting of: SOX1, SOX2, SOX3, SOX15, SOX18, KLF1, KLF2, KLF4, KLF5, C-MYC, L-MYC, N-MYC, SALL4, OCT4, NANOG, STELLA, Esrrb, NOBOX, STAT family members FoxD3, UTF1, Rex1, ZNF206, Myb12, DPPA2, ESG1, Otx2 and LIN28, and any combination thereof.

26. The method of claim 22, wherein the expression cassette comprises four genes that induce pluripotency.

27. The method of claim 26, wherein the genes are OCT4, SOX2, KLF4 and optionally C-MYC.

28. The method of claim 18, wherein the selectable marker is a gene selected from the group consisting of: neomycin resistance gene, puromycin resistance gene, guanine phosphoribosyl transferase, dihydrofolate reductase, adenosine deaminase, puromycin-N-acetyltransferase, hygromycin resistance gene, multidrug resistance gene, or hisD gene.

29. The method of claim 28, wherein the selectable market is the hygromycin resistance gene.

30. The method of claim 17, wherein the first and third polynucleotide sequences have a length of between about 0.5 kb and 5 kb.

31. The method of claim 30, wherein the first polynucleotide sequence has a length of about 3.5 kb.

32. The method of claim 30, wherein the third polynucleotide sequence has a length of about 2.6 kb.

33. The method of claim 17, wherein the promoter is a cytomegalovirus (CMV) promoter.

34. The method of claim 17, wherein the translation initiation site is an internal ribosome entry site (IRES).

35. The method of claim 17, wherein the introduction of the nucleic acid construct into the somatic cell is non-viral based.

36. The method of claim 17, wherein the nucleic acid construct is introduced into the somatic cell by electroporation, calcium phosphate mediated transfer, nucleofection, sonoporation, heat shock, magnetofection, liposome mediated transfer, microinjection, microprojectile mediated transfer, cationic polymer mediated transfer, or cell fusion

37. An induced pluripotent stem (iPS) cell produced using the method of claim 17.

38. A population of induced pluripotent stem (iPS) cells produced using the method of claim 17.

39. A method of treating a subject comprising:
a) obtaining a somatic cell from a subject;
b) reprogramming the somatic cell into an induced pluripotent stem (iPS) cell using the method of claim 1;
c) culturing the pluripotent stem (iPS) cell to differentiate the cell into a desired cell type suitable for treating a condition; and
d) introducing into the subject the differentiated cell, thereby treating the condition.
